(19) United States
(12) Patent Application Publication
(10) Pub. No.: US 2017/0110019 A1
(43) Pub. Date: Apr. 20, 2017

(54) Apparatus and Method for Brain Training

(71) Applicant: Fung Ling Chow, Kowloon East (HK)

(72) Inventor: Fung Ling Chow, Kowloon East (HK)

(21) Appl. No.: 14/918,477

(22) Filed: Oct. 20, 2015

(51) Int. Cl. G09B 5/06; G09B 19/00; A61B 5/0484

(52) U.S. Cl. CPC G09B 5/06; A61B 5/0484; G09B 19/00

(57) ABSTRACT

An apparatus and method for brain training, including at least one first vibratory stimulation unit configured to generate vibratory stimuli with the user's skull; at least one second vibratory stimulation unit configured to generate vibratory stimuli with the user's body movement senses; at least one brain wave detecting unit configured to detect the brain wave of the user; and a controlling unit configured to connect with and control the visual stimulation unit, the first vibratory stimulation unit, the second vibratory stimulation unit and the brain wave detecting unit. By using the apparatus, the user can trigger active different parts area of brain to respond to the stimulations in a non-invasive way to improve the metabolism of brain tissue and help the neurons-to-neurons communication, which would be used in different occasions to improve the learning ability of users.

### FIELD OF THE INVENTION

The present invention relates to an apparatus and a method for brain training.

### BACKGROUND OF THE INVENTION

The human brain is made up of an uncountable amount of neurons, which all communicate with each other via electrical impulses. These impulses result in waves, which can be detected and measured with an electroencephalography (EEG). Among all the kinds of brain waves, researches show that alpha brain wave has a posiptive effect on learning speed and memorization.

Memorization is a necessary skill for everyone, no matter in education, living or working. However, it is hard for some kinds of people, such as the ones who suffered serious disease like stroke, senile dementia, learning disabilities, to memorize effectively.

### SUMMARY OF THE INVENTION

The problem to be solved in the present invention is to provide an apparatus and a method for brain training effectively in a non-invasive way.

The present invention provides an apparatus for brain training, comprising:

a first vibratory stimulation unit adapted for generating a first vibratory stimulus with the user's head;

a second vibratory stimulation unit adapted for generating a second vibratory stimulus with the user's body movement senses;

a brain wave detecting unit adapted for detecting the brain wave of the user's brain functioning; and

a controlling unit adapted for controlling said first vibratory stimulation unit, said second vibratory stimulation unit and said brain wave detecting unit.

The present invention provides a method for non-invasive brain training, wherein said first vibratory stimulation unit further generates an auditory stimulus on the user through inner ear-bone, comprising the steps of:

Step 101: stimulating a user simultaneously by a first combination of said auditory stimulus, said first vibratory stimulus and said second vibratory stimulus, wherein the contents of said first combination come from said controlling unit;

Step 102: monitoring the user's brain wave to detect pre-defined brain wave;

Step 103: if pre-defined brain wave is detected, stimulating the user simultaneously by a second combination of said auditory stimulus, said first vibratory stimulus and said second vibratory stimulus, wherein the contents of second combination includes the contents of the first combination of step 101; and

Step 104: confirming the change of said second combination from said first combination with body movement by the user.

The present invention provides another method for non-invasive brain training that includes a visual stimulation unit that generates a visual stimulus, wherein said first vibratory stimulation unit further generates an auditory stimulus on the user through inner ear-bone, comprising the steps of:

Step 201: stimulating a user simultaneously by a first combination of visual stimulus, said auditory stimulus, said first vibratory stimulus and said second vibratory stimulus, wherein the contents of said first combination come from said controlling unit;

Step 202: repeating the stimulation of the combination of said auditory stimulus, said first vibratory stimulus and said second vibratory stimulus of Step 201 to set up neurons-to-neurons connection for memorization;

Step 203: urging the user to repeat the content of said auditory stimulus of step 201 orally; and

Step 204: urging the user to describe the content of said visual stimulus of step 201 orally.

By using the apparatus and method, the user can active different positions of brain to respond to the stimulations in a non-invasive way to improve the metabolism of brain tissue and enhance the neurons-to-neurons communication, which would be used in different occasions to improve the learning ability of users.

### BRIEF DESCRIPTION OF THE DRAWINGS

This and other objects, features and advantages of the invention will become apparent upon reading of the following detailed descriptions and drawings, in which:

FIG. 1 is a top view of the cerebral cortex's partition of an embodiment of the present invention;

FIG. 2 is a top view of the cerebral cortex's partition of another embodiment of the present invention;

FIG. 3 is an enlarged view of bone conductor and brain wave electric poles of the present invention;

FIG. 4 is an alternative perspective view of FIG. 3;

FIG. 5 is a schematic of the second vibratory stimulation elements of the present invention;

FIG. 6 is a circuit schematic of a bone conductor of the present invention;

FIG. 7 is a circuit schematic of a switch controller of the present invention;

FIG. 8 is a circuit schematic of an embodiment of the present invention;

FIG. 9 is the training process of an embodiment of the present invention.

### DESCRIPTION OF THE PREFERRED EMBODIMENTS

The present invention provides an apparatus and a method for brain training in a non-invasive way by stimuli of a combination of hearing, skull vibratory and body vibratory movement senses. Body movement senses is mind reaction processing. As user is perceived the disturbance of mind by external vibratory stimuli acting on skull, inner ear and muscle of body, he/she motivates body movement senses to react these stimuli as brain, eyes, mouth and body move as finger pressed button etc. The present invention can be used in different occasions to improve the learning ability of users.

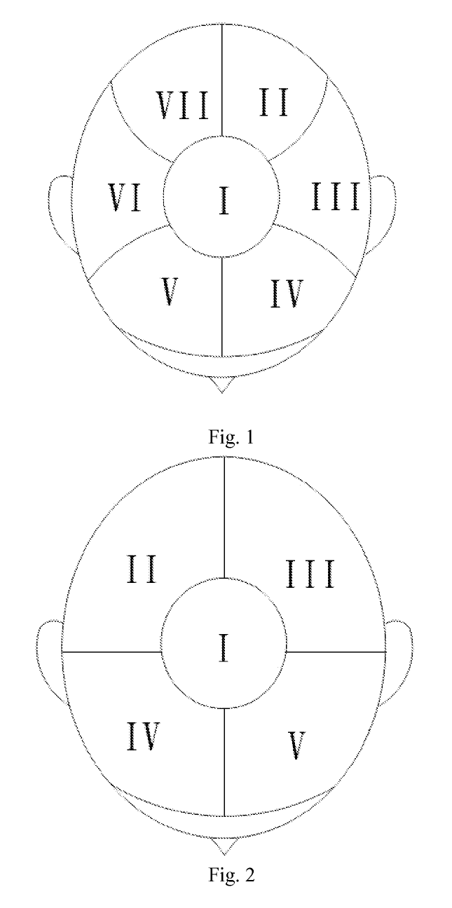

FIGS. 1 and 2 separately show partitions of the user's cerebral cortex in top view. It is common knowledge for person having ordinary skill in the art that cerebral cortex can be divided into different function areas, such as motion area, verbal area, and cognition area. The detailed partition method can be varied from case to case for the application of the present invention. Referring to FIG. 1, the user's cerebral cortex can be divided into 7 parts area while referring to FIG. 2, the user's cerebral cortex can be divided into 5 parts area. As such, the detailed partition method of the cerebral cortex does not limit the present invention and the following embodiments will be based on FIG. 1, which means the user's cerebral cortex would be treated as divided into 7 parts area. And as the present invention is applied in a non-invasive way, the division of cerebral cortex can be treated as the division of skull, or the division of head in practical use.

In an embodiment, the apparatus of the present invention includes a controlling unit 1, seven first vibratory stimulation units 2 to generate vibratory stimuli with the user's cerebral cortex; seven second vibratory stimulation units 3 to generate vibratory stimuli with the user's fingers; and one brain wave detecting unit 4 to detect the brain wave of the user. When the user uses the apparatus, he/she will feel three kinds of senses including hearing, skull vibratory and body vibratory senses at the same time, which would induce alpha brain wave and improve the learning ability of user eventually.

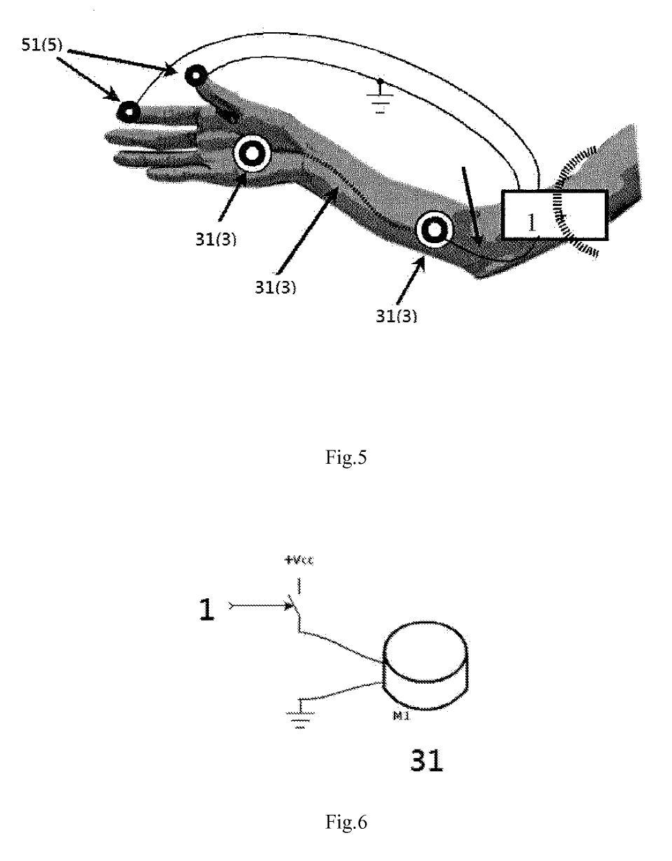

The controlling unit 1 is used to receive instruction from user and coordinate the running of the apparatus. The controlling unit 1 connects separately with the first vibratory stimulation unit 2, the second vibratory stimulation unit 3 and brain wave detecting unit 4. Preferably, the controlling unit 1 connects with the other parts of the apparatus in a wireless way, such as via blue tooth or infrared ray. In some embodiments, when the first vibratory stimulation units 2 are attached together as helmet-like which can be worn on the user's head (not shown), the controlling unit 1 can be embedded in the helmet-like. While in other embodiments, the controlling unit 1 can be individually placed on the user's body, such as the user's arm as shown in FIG. 5.

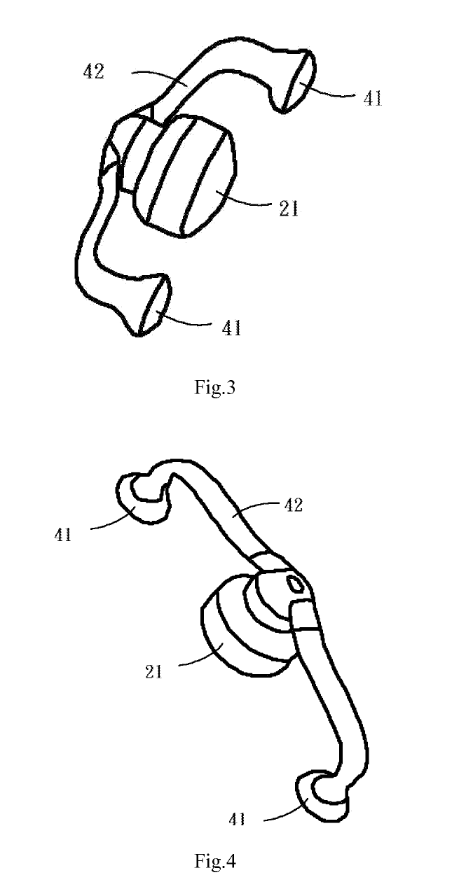

The first vibratory stimulation unit 2 includes at least one bone conductor 21, referred as FIG. 3 and FIG. 4. The sizes or shapes of the bone conductor 21 do not limit the present invention. Bone conduction is the conduction of sound-vibrating to act on the inner ear-bone through the bones of the skull and bone conduction transmission can be used with individuals with normal or impaired hearing. To be specific, the bone conductor 21 is generated by the audio input signals of a first auditory stimulation element 22. Preferably, the bone conductor 21 can also be presented by another audio input signals of a second auditory stimulation element 23, which can be referred as FIG. 8. During the application of the apparatus, if pre-defined brain wave occurs, which means alpha brain wave occurs here, the second auditory stimulation element 23 will be triggered switch by the alpha brain wave signals which is in electrical pulse to generate the vibration of bone conductor 21. The content of the second auditory stimulation element 23 can be mixed with the content of the first auditory stimulation element 22, or can be mixed with the content of first auditory stimulation element 22 and the detected alpha brain wave, which may be presented as irregular noise. As such, the user will hear different sound or more sound information from the previous sound generated by the first auditory stimulation element 22. And the user would be able to hear how his brain wave sounds. More preferably, the content of the second auditory stimulation element 23 is mixed by an audio mixer 24 as shown in FIG. 8.

Referring to FIG. 5, the second vibratory stimulation unit 3 includes at least one micro vibration motor 31 which are laid on the user's fingers. In other embodiments, the micro vibration motors 31 can be laid on limbs such as arms, legs, palms, or other parts of the user's body. FIG. 6 shows the circuit diagram of the micro vibration motor 31. The controlling unit 1 outputs some specified signals to connect the circuit of the micro vibration motor 31 which would generate the vibration of the micro vibration motor 31. The actual size or the vibration level of the micro vibration motor 31 does not limit the present invention. Preferably, the number of the micro vibration motor 31 is same as that of the division of skull such that stimulation in different parts area of cerebral cortex can be in accordance with different body vibration stimulation. For example, the stimulation in section I shown as FIG. 1 can be in accordance with the vibratory stimulation on the user's palm, which help the user better focus on the specified positions of his/her brain area.

The brain wave detecting unit 4 includes at least one set of brain wave electric poles 41. Preferably, every bone conductor 21 connects with two brain wave electric poles 41 by a connecting element 42 to better detect the raw brain wave signal, referred as FIG. 3. Further, as the raw brain wave is generally too weak to indicate or identify, amplifier and filter for noise reduction would be necessary to identify alpha brain waves. Hence in some embodiments, the brain wave detecting unit 4 further includes a first amplifier 43 and a first stage filter 44 for noise reduction. The brain wave electric poles 41 keep touch with the user's head to detect the raw brain wave signal of user and are connected with the first amplifier 43. After amplified by the first amplifier 43, the detected brain wave signal will be filtered by the first stage filter 44 which is connected with the first amplifier 43 to reduce noise, or some unexpected signals. More preferably, referred to FIG. 8, the brain wave detecting unit 4 further includes a second amplifier 45 and a second stage filter 46, and other elements such as inductor 47, optical isolator 48. The filtered signal will be further amplified by the second amplifier 46 and further filtered by the second stage filter 45 to identify alpha brain wave effectively. To be specific as referred in FIG. 8, the first stage filter 44 is a filter capacitor and the second stage filter 46 is a combination of a low pass filter circuit which cuts off at 33.9 Hz and an alpha wave pass filter.

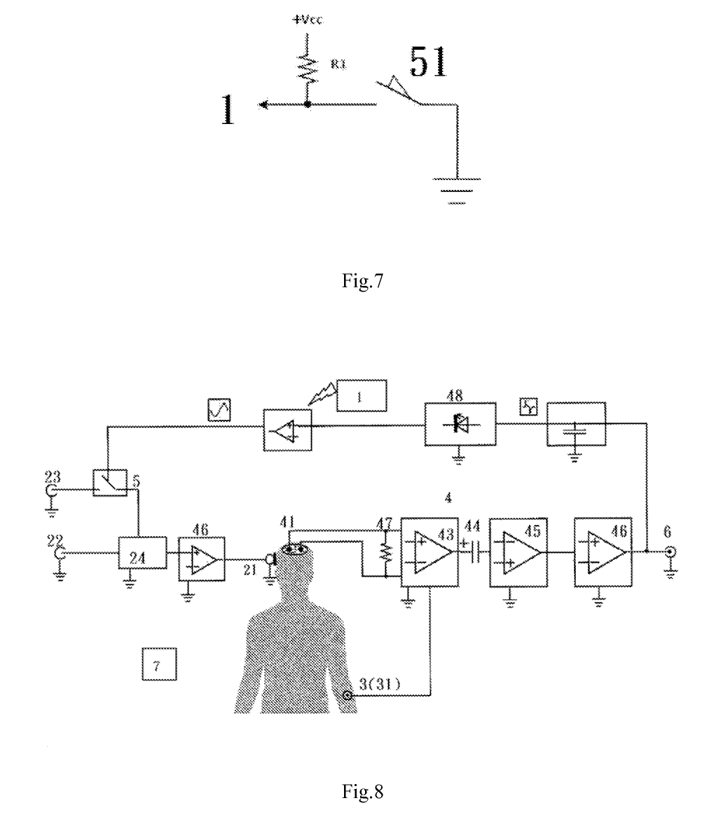

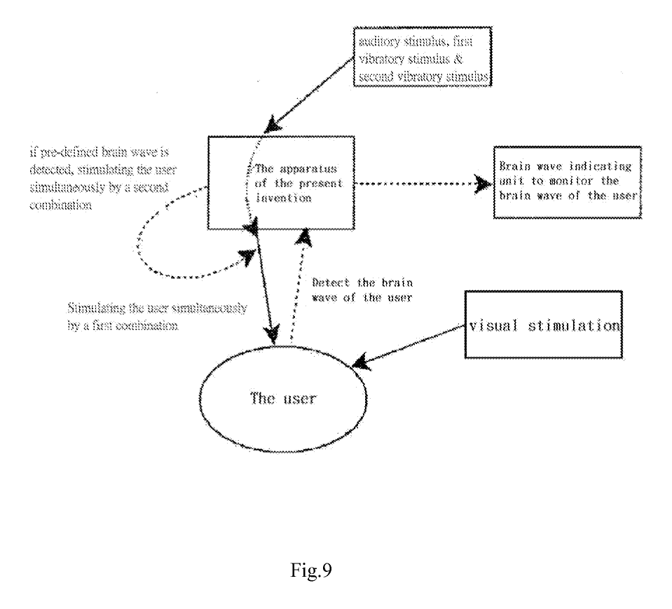

In other embodiment, referring to FIG. 5, the apparatus further includes a switch controlling unit 5 to switch the active first vibratory stimulation unit 2 and brain wave detecting unit 4. In this embodiment, the switch controllers 51 are attached to the users' fingers. Referred as FIG. 5, a switch controller 51 includes two contacts. One contact is attached to the user's index finger while another contact is attached to the user's thumb. The user can move his/her index finger or thumb to close the contacts which will switch the active first vibratory stimulation unit 2 and brain wave detecting unit 4. Preferably, the switch controlling unit 5 includes more than one switch controller 51. The number of switch controller 51 matches that of the first vibratory stimulation units 2. Alternatively, the number of switch controller 51 matches that of vibratory stimulation units 2. For example, when the number of first vibratory stimulation units 2 is seven as stated in the previous embodiments, the number of switch controllers 51 can be nine in total, which includes two additional public switch controllers, or switch contacts. The public switch contacts are used to control more than one vibratory stimulation units. When there are two public switch contacts, each public switch contact controls half numbers of the vibratory stimulation units, which means one public switch contact controls the vibratory stimulation units on the left brain while the other public switch contact controls the vibratory stimulation units on the right brain. More preferably, the switch controller 51 is a multiplexer. FIG. 7 shows the circuit diagram of the switch controller 51. The user can switch on or off the switch controller 51. When the switch controller 51 is switched on, the circuit will be connected and signal out some specified signal to the input end of the controlling unit 1 to control the specified first vibratory stimulation unit(s) 2 and second vibratory stimulation unit(s) 3.

In another embodiment, the apparatus further includes a brain wave indicating unit 6. The brain wave indicating unit 6 can be a display monitor, a personal computer or indicator light. Take indicator light for example. When alpha brain wave is identified by the brain wave detecting unit 4, the indicator light would switch from one status to another status to present, or alert the occurrence of alpha brain wave. For example, the indicator light would turn on from the status of turn-off to alert the occurrence of alpha brain wave. Alternatively, the indicator light would turn red to alert the occurrence of alpha brain wave. The detailed alert method of the brain wave indicating unit 6 does not limit the present invention.

Take the learning process of the character ‘A’ of a stroke patient to explain the operation of the apparatus. When the controlling unit 1 receives the instruction, it would communicate with the first vibratory stimulation unit 2 to generate the vibratory on the user's skull, which generates hearing effects and sense of skull vibration to the user. Meanwhile, the controlling unit 1 would communicate with the second vibratory stimulation unit 3 to generate vibratory on the user's body. As such, the user would be stimulated by a combination of hearing, skull vibratory and body vibratory movement senses. During the stimulation period, the brain wave of the user would be monitored simultaneously by the brain wave detecting unit 4 to detect the occurrence of alpha brain wave, which indicates a successful neurons-to-neurons communication for memorization. If alpha brain wave is detected, the second auditory stimulation element 23 would be activated to generate the vibratory on the user's skull, whose content is mixed with that of the previous vibratory on the user's skull. Alternatively, the content of second auditory stimulation element 23 is mixed with that of the previous vibratory and detected alpha brain wave. The user confirms the change of what he hears by pressing down the switch controlling unit 5, which is a feedback from the user after the brain training process showing successful neurons-to-neurons communication. As such, the first vibratory stimulus from the first vibratory stimulation unit 2 and second vibratory stimulus from the second vibratory stimulation unit 3 enhance the communication among nerve tissues, which brings the change of brain waves frequency indicating the current brain parts area be activated. The user exercises his/her limb, such as pressing down the according switch controller in response to the receipt of the first and second vibratory stimulus. Also, the user is able to switch one stimulated parts area to another parts area to train other parts area of his/her brain. By repeating the training combination of skull vibratory and limb vibratory, it would be possible to construct a neuron-to-neuron loop to control the user's limb, or remodeling the user's brain loop in an artificial method. Thus, the apparatus of the present invention can help the neurons-to-neurons communication to prevent memory loss for the user. Also, the apparatus of the present invention can help stroke patients recover by delaying brain degeneration. And the user can be alert in time by observing the brain wave indicating unit 6, which is especially useful for patients who suffered serious disease such as stroke.

During the application of the apparatus of the present invention, as there are more than one first vibratory stimulation unit 2 and second first vibratory stimulation unit 3, the user can trace vibratory feelings in different parts area according to his own senses, which would enhance neurons-to-neurons communication to accelerate metabolism of the damaged nerve tissues for recovery.

In other embodiments, the apparatus can further include a visual stimulation unit 7 to generate visual stimuli, which would be useful not only for patients who suffered serious brain disease, but also for students who need to memorize efficiently. The visual stimulation unit 7 can be any display device, such as a detachable LED monitor. The visual content can be text, video or picture, which is input by controlling unit 1. The playing speed and/or playing mode of the visual content can be altered by the controlling unit 1, such as playing a set of texts every 15 minutes or playing a video in a slow-down speed. Take the above learning process of the character ‘A’ for example. When a nurse who takes care of the user inputs the character ‘A’ into the controlling unit 1, the visual stimulation unit 7 will display the character ‘A’. Meanwhile, the first auditory stimulation element 22 will generate the vibration of bone conductor 21 so that the user will hear the pronunciation of the character ‘A’ and feel the vibration of skull. Also, the user will feel the vibration of his body. As the neuron works individually and it would be difficult for the brain to learn the content in one time, visual stimuli and vibration stimuli may repeatedly stimulate cerebral cortex until the user realizes the connection between what he sees and what he hears. The use of bone conductor 21 will be in accordance with the visual stimulation unit 7, which can be achieved by varying in frequency and duration and so on. For example, the frequency or duration of the character ‘A’ is different from that of the character 13′ so that user can identify ‘A’ from ‘B’ by using bone conductor. Also, the bone conductor 21 should be altered by the controlling unit 1 to comply with the visual stimulation unit 7 so as to establish the visual and auditory connection in brain.

A method for brain training of the present invention which is especially for patients who suffered from stroke, in which the first vibratory stimulation unit 2 further generates an auditory stimulus on the user through inner ear-bone, including:

Step 101: stimulating a user simultaneously by a first combination of said auditory stimulus, said first vibratory stimulus and said second vibratory stimulus, wherein the contents of said first combination come from said controlling unit;

Step 102: monitoring the user's brain wave to detect pre-defined brain wave;

As stated above, alpha brain wave has a positive effect on learning speed and memorization. Thus, the pre-defined brain wave is alpha brain wave.

Step 103: if pre-defined brain wave is detected, stimulating the user simultaneously by a second combination of said auditory stimulus, said first vibratory stimulus and said second vibratory stimulus, wherein the contents of second combination includes the contents of the first combination of step 101;

In other embodiment, the content of the second auditory stimulation can further includes brain wave of the user.

Step 104: confirming the change of the second combination from the first combination with body movement by the user.

There are various ways to confirm the change of the second combination from the first combination by a user. For example, when switch controller 51 is laid on the user's fingers, shown as FIG. 5, the user can press down the switch controller 51 by fingers.

By using the method provided as above, the user can be helped to set up the controlling of brain tissue to body in a non-invasive way, which would greatly improve the recovery of patients who suffered from stroke.

A method for brain training of the present invention which is especially for students that includes a visual stimulation unit that generates a visual stimulus, in which the first vibratory stimulation unit 2 further generates an auditory stimulus on the user through inner ear-bone, including:

Step 201: stimulating a user simultaneously by a first combination of visual stimulus, the auditory stimulus, the first vibratory stimulus and the second vibratory stimulus, in which the contents of the first combination come from the controlling unit;

Visual staying phenomenon happened when people observe rapid movement objects. Generally, brain would remember the visual content in the latest 1/24 second. Reproduction of the visual content in the latest 1/24 second indicates memorization has been successful for the user. Visual stimulation combined with the skull vibratory stimulation, auditory stimulation and body vibratory stimulation would induce the reproduction of the visual content in the latest 1/24 second in less time, which means greatly improve memorization ability.

Step 202: repeating the stimulation of the combination of said auditory stimulus from the first vibratory stimulus, the first vibratory stimulus and the second vibratory stimulus of Step 201 to set up neurons-to-neurons connection for memorization;

Step 203: urging the user to repeat the content of the auditory stimulus of step 201 orally; and

Step 204: urging the user to describe the content of said visual stimulus of step 201 orally.

By using the method provided as above, the user can activate different parts area of brain to respond to the stimulations in a non-invasive way to improve the metabolism of brain tissue and help the neurons-to-neurons communication which would be used in different occasions to improve the learning ability of users.

The present invention has been described in detail, with reference to the preferred embodiment, in order to enable the reader to practice the invention without undue experimentation. However, a person having ordinary skill in the art will readily recognize that many of the previous disclosures may be varied or modified somewhat without departing from the spirit and scope of the invention. Accordingly, the intellectual property rights to this invention are defined only by the following claims.

1. An apparatus for brain training for a user, comprising:
at least one first vibratory stimulation unit for generating a first vibratory stimulus with the user's head;
at least one second vibratory stimulation unit for generating a second vibratory stimulus with the user's body movement senses;
a brain wave detecting unit for detecting the brain wave of the user's brain functioning; and
a controlling unit for controlling said first vibratory stimulation unit, said second vibratory stimulation unit and said brain wave detecting unit.

2. The apparatus in accordance with claim 1, wherein said apparatus further comprises a visual stimulation unit for generating a visual stimulus, said controlling unit further controls said visual stimulation unit.

3. The apparatus in accordance with claim 2, wherein said apparatus further comprises a switch controlling unit for switching on and off said first vibratory stimulation unit, said second vibratory stimulation unit and/or said brain wave detecting unit.

4. The apparatus in accordance with claim 3, wherein said switch controlling unit comprises at least one switch controller, the number of said switch controller is in accordance with the number of said first vibratory stimulation unit.

5. The apparatus in accordance with claim 3, wherein said switch controlling unit comprises a multiplexer for switching on and off said first vibratory stimulation units, said second vibratory stimulation unit and/or said brain wave detecting units.

6. The apparatus in accordance with claim 3, wherein said apparatus further comprises at least one brain wave indicating unit for indicating the brain wave of user.

7. The apparatus in accordance with claim 6, wherein the number of brain wave indicating units is in accordance with the number of said first vibratory stimulation units.

8. The apparatus in accordance with claim 6, wherein said first vibratory stimulation unit comprises a first auditory stimulation element and a second auditory stimulation element; said first auditory stimulation element for generating a first auditory signal; said second auditory stimulation element for generating a second auditory signal whose content includes said first auditory signal; said second auditory stimulation element generates said second auditory signal after receipt of the pre-defined brain wave signal stimulated by said first auditory stimulation element.

9. The apparatus in accordance with claim 8, wherein said second auditory stimulation element for generating a second auditory signal whose content includes said first auditory signal and the brain wave detected by said brain wave detecting unit.

10. The apparatus in accordance with claim 9, wherein said pre-defined brain wave signal is alpha brain wave.

11. The apparatus in accordance with claim 8, wherein said brain wave detecting unit comprises a brain wave electric pole, a first amplifier and a first stage filter;
said brain wave electric pole is for detecting the raw brain wave signal of the user which is stimulated by first auditory stimulation element and is connected with said first amplifier;
said first amplifier is for amplifying the raw brain wave signal detected by said brain wave electric pole and is connected with said brain wave electric pole and said first filter separately; and
said first stage filter is for filtering the brain wave which has been amplified by said first amplifier.

12. The apparatus in accordance with claim 10, wherein said brain wave detecting unit further comprises a second amplifier and a second stage filter;
said second amplifier is for amplifying the brain wave from said first stage filter;
said second amplifier is connected with said first stage filter and said second stage filter separately; and
said second stage filter is for filtering the brain wave from unexpected signals of said second amplifier.

13. The apparatus in accordance with claim 1, wherein said first vibratory stimulation unit is a bone conductor; and said second vibratory stimulation unit is a micro vibration motor.

14. The apparatus in accordance with claim 4, wherein said switch controller includes two contacts which are adapted to attach on different fingers of the user; when the two contacts are closed, the circuit of said switch controller is connected to switch on the active first vibratory stimulation unit and brain wave detecting unit.

15. The apparatus in accordance with claim 13, wherein said apparatus comprises seven bone conductors and seven micro vibration motors; said seven bone conductors are laid on different areas of the user's head and said seven micro vibration motors are attached on different areas of the user's limb.

16. A method for brain training using an apparatus in accordance with claim 1, wherein said first vibratory stimulation unit further generates an auditory stimulus on the user through inner ear-bone, comprising the steps of:
Step 101: stimulating a user simultaneously by a first combination of said auditory stimulus, said first vibratory stimulus and said second vibratory stimulus, wherein the contents of said first combination come from said controlling unit;
Step 102: monitoring the user's brain wave to detect pre-defined brain wave;
Step 103: if pre-defined brain wave is detected, stimulating the user simultaneously by a second combination of said auditory stimulus, said first vibratory stimulus and said second vibratory stimulus, wherein the contents of second combination includes the contents of the first combination of step 101; and
Step 104: confirming the change of said second combination from said first combination with body movement by the user.

17. The method in accordance with claim 16, wherein the content of said second combination further includes the brain wave of the user.

18. The method in accordance with claim 17, wherein said body movement is pressing down a switch by hand.

19. The method in accordance with claim 18, wherein said pre-defined brain wave is alpha brain wave.

20. The method for brain training using an apparatus in accordance with claim 2, wherein said first vibratory stimulation unit further generates an auditory stimulus on the user through inner ear-bone, comprising the steps of:
Step 201: stimulating a user simultaneously by a first combination of visual stimulus, said auditory stimulus, said first vibratory stimulus and said second vibratory stimulus, wherein the contents of said first combination come from said controlling unit;
Step 202: repeating the stimulation of the combination of said auditory, said first vibratory stimulus and said second vibratory stimulus of Step 201 to set up neurons-to-neurons connection for memorization;
Step 203: urging the user to repeat the content of said auditory stimulus of step 201 orally; and
Step 204: urging the user to describe the content of said visual stimulus of step 201 orally.
